(19) United States
(12) Patent Application Publication
(10) Pub. No.: US 2023/0067402 A1
(43) Pub. Date: Mar. 2, 2023

(54) ISOTOPICALLY LABELED TRYPTAMINES AND ANALOGS THEREOF

(71) Applicant: Biomind Labs Inc, Pando (UY)

(72) Inventors: Lucía Fernanda Minini Rivas, Montevideo (UY); Fernando Amaury Ferreira Chiesa, Montevideo (UY); Dráulio Barros de Araújo, Natal (BR); Paola Alexandra Díaz Dellavalle, Montevideo (UY)

(73) Assignee: Biomind Labs Inc, Pando (UY)

(21) Appl. No.: 17/887,109

(22) Filed: Aug. 12, 2022

Related U.S. Application Data
(60) Provisional application No. 63/234,035, filed on Aug. 17, 2021.
(60) Provisional application No. 63/234,038, filed on Aug. 17, 2021.

(51) Int. Cl. C07D 209/16

(52) U.S. Cl. CPC C07D 209/16; C07B 2200/05

(57) ABSTRACT

The present invention discloses compounds of formula (I)

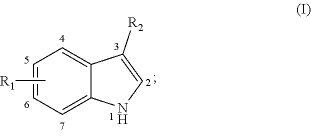

wherein R₁ is H, OH, PO₄H₂, OCH₃, or SCH₃; wherein R₂ is H or

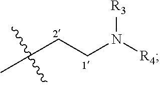

and wherein R₃ and R₄ are H or CH₃; and the compound of formula (I) has one or more of a ¹⁵N atom and/or a ¹³C atom. Also disclosed are methods of making the compounds of formula (I), as well as pharmaceutical compositions containing a compound of formula (I) and DMT or 5-MeO-DMT.

### CROSS REFERENCE TO RELATED APPLICATIONS

This application claims priority to U.S. Provisional Application 63/234,035 filed Aug. 17, 2021 and U.S. Provisional Application 63/234,038 filed Aug. 17, 2021, of which the entire contents of each are incorporated by reference.

### FIELD OF THE INVENTION

The present invention relates to isotopically labeled tryptamines and analogs thereof, methods for their preparation, and compositions thereof. The tryptamines and analogs thereof are isotopically labeled with ¹³C and/or ¹⁵N at positions in the indole ring or on the alkylamine side chain.

### BACKGROUND OF THE INVENTION

Classic psychedelics have been used by humans for thousands of years, with a strong tradition in indigenous cultures where they were used in healing events, rituals, and sacramentals. Despite this, research on their pharmacological use only begun to emerge significantly in the 1990s. Recent clinical trials have suggested that these substances would be safe and effective in their use in the treatment of several mental health conditions with unmet needs (Nichols et al.).The therapeutic effects of psychedelics depend on the psychedelic experience felt by the patient during treatment (Bogenschutz et al.; Garcia-Romeu et al.; Ross et al.; Palhano-Fonteset al.) Studies suggest that elements of the psychedelic experience mediate the therapeutic benefit.

Classic psychedelics include tryptamine derivatives, such as psilocybin, psilocin, N,N-dimethyltryptamine (DMT), 5-MeO-N,N-dimethyltryptamine (5-MeO-DMT), bufotenin, baeocystin and norbaeocystin. These derivatives have a pharmacological action mechanism based on their agonist action on the serotonin 5HT2A receptor (Chi, T. A.). DMT is an indole alkaloid that is widely distributed in plants and animals, which was first synthesized in 1931 by Canadian chemist Richard Manske, and isolated from the root bark of Mimosa tenuiflora in 1946 by Brazilian chemist Oswaldo Lima (Lima, O. G.). 5-Methoxy-N,N-dimethyltryptamine (i.e., 5-MeO-DMT) is an indole alkaloid that shares structural and pharmacological similarities with the DMT, and has attracted increasing interest in the scientific community (David, E. N.). 5-MeO-DMT appears to have positive effects on mental disorders, such as depression and anxiety (Davis et al. (1)). In Central and South America the main sources of 5-MeO-DMT are Anadenanthera peregrina (yopo or cohoba) and Virola theiodora, in addition to the glandular secretions of the Sonoran Desert toad (Bufo alvarius),which is native to the southwestern United States and northwestern Mexico.

Clinical studies of tryptamines with hallucinogenic effects include treatment of various neurological pathologies, such as depressive disorders, anxiety disorders, substance abuse, and nervous anorexia (Palhano-Fontes et al.; Sanches et al.; Osório et al.). These studies have shown that the use of these tryptamine derivatives generates benefits for the patient in the disorders mentioned above. The studies are mainly focused on the use of psilocybin, and on compositions containing DMT together with inhibitors of the enzyme monoamine oxidase (i.e. MAO), for example, ayahuasca (Luna, L. E.; Johnson et al.).

Within the classic psychedelics with good profiles for the treatment of psychiatric disorders, the use of the tryptamine alkaloids DMT and its analog 5-methoxy-N,N-dimethyltryptamine (5-MeO-DMT) are highlighted because they act and are eliminated quickly (Sanches et al.). This quick action is due in part to the high rate of metabolization they experience regardless of the route of administration, such as intravenous or intramuscular. Once these compounds are administered, only traces can be found in the urine of the patient. The administration of DMT intramuscularly reaches a peak concentration in the blood within the first 10-15 minutes of its administration and goes to undetectable levels after one hour. A fundamental aspect of DMT pertinent for treatment is that it is only active orally if co-administered with an inhibitor of the enzyme monoamine oxidase (iMAO) DMT is greatly affected on first passage through the liver by action of the enzyme monoamine oxidase (MAO) (Baker, S. A.), which is present in the gastrointestinal tract and makes DMT inaccessible to the circulatory system and central nervous system.

Traditionally, DMT is consumed for medicinal purposes by indigenous populations of the Amazon Basin in a drink known as Ayahuasca. This drink manages to neutralize the action of MAO enzymes, allowing the DMT to take action in the central nervous system (Luna, L. E.). This is achieved because it is prepared by the decoction of two plants, leaves of Psychotria viridis, which contain DMT, and the bark of the Banisteriopsis caapi vine, which is rich in iMAOs such as harmine and harmaline (Riba et al., Mckenna et al.). In some cases, traditional preparations use other vegetable species that contain active compounds with similar biochemical properties (Kaasik et al.).

Preclinical and clinical trials with Ayahuasca have suggested a therapeutic potential for the treatment of psychiatric disorders with promising evidence of rapid antidepressant effects in patients with treatment-resistant depression, with effects observed as early as 24-hours after treatment (Palhano-Fontes et al.; Sanches et al.; Osório et al.). In a clinical study with 17 patients, a quick and significant antidepressant clinical effect was observed and evaluated 40 minutes after the ingestion of Ayahuasca and the symptoms were significantly reduced over 21 days. Neuro-imaging examination (SPECT) was performed 8 hours after treatment, and increases in blood perfusion were observed in brain regions involved in the regulation of mood and emotions, such as the nucleus accumbens, the insula, and the anterior cingulate (Sanches et al).

The results were confirmed in a randomized placebo-controlled trial which evaluated 218 patients with depression, of whom 35 were selected. Significant antidepressant effects were observed one day after the ayahuasca session, and persisted for at least 7 days. These effects were statistically superior to the effects seen in the placebo group. Response rates of 50%, 77% and 64% were found, one, two and seven days after the administration of ayahuasca, respectively (Palhano-Fontes et al.).

Despite good drug use profiles, psychedelic-based treatments are disfavored due to individual variability of the hallucinogenic experience and its corresponding consequences on the therapeutic efficacy of the treatment. The variability of the hallucinogenic experience between people is largely due to differences in the individual's rate of metabolization of tryptamines. These differences in metabolization are a consequence of many factors, such as speed of drug distribution and degradation, and impacts the physiological, biological, and molecular effects of the tryptamines. Naturally, with psychedelic substances, an individual's rate of metabolism will impact the quality of the acute psychedelic experience which in turn influences the therapeutic outcome. In addition, genetic factors are involved, since this process is driven by enzymes and transportation proteins which affects the rate at which a drug is transported into cells, its breakdown within cells, and its excretion. Environmental factors can also influence the individual's metabolism. For example, an individual's diet may increase the production of a relevant enzyme or provide cofactors that modulate the activity of different enzymes, particularly when given orally or inhaled (Callaway, J. C.).

The primary pathway of metabolization for DMT is degradation by the enzyme monoamine oxidase (i.e. MAO), which produces 3-indoleacetic acid (IAA; structure A below) as its main metabolite. The secondary metabolites that are formed include DMT-N-oxide (DMT-NO; structure B below), the most abundant secondary metabolite, and low amounts of N-methyltryptamine (NMT; structure C below). The primary metabolite, IAA, can also come from processing of DMT precursors, such as tryptamine and N-methyltryptamine, via MAO (Baker, S. A.).

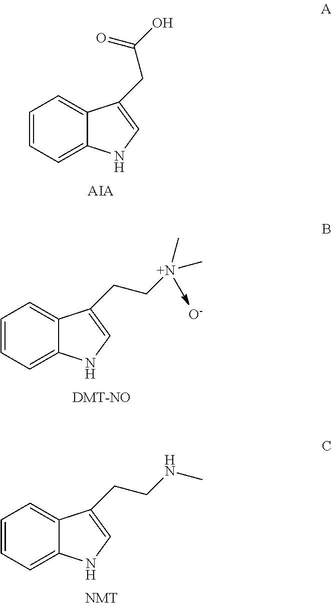

Therefore, the presence of endogenous DMT and its metabolites, mainly the IAA, generates an additional difficulty in DMT's use as a pharmaceutical drug because it is not possible to carry out a study of the pharmacokinetics of the compounds that allow one to know the correct bioavailability and elimination of the drug and thus make an adequate adjustment of the dose in an eventual treatment.

### SUMMARY

There is thus a need for compounds that allows one to follow the drug after administration and its metabolites in order to determinate bioavailability. Isotopic labelling represents a strategy to follow the metabolization of these compounds and allow for their monitoring.

*to be completed once claims agreed to*

### DETAILED DESCRIPTION

The main objective of medical laboratories is to provide information that is useful to assist in the decision-making process in treatments, allowing for optimization of health care. This can only be achieved when reliable analytical results are obtained from the patient sample. Errors in these areas can lead to incorrect interpretations by the physician, which could lead to undesirable consequences for medical practice, health care systems, and the patient. One of the biggest problems is comparability between analytical results that originate in different laboratories using different analytical methods for the same analyte (Panteghini, M.).

To properly evaluate drugs, it is preferred to have methodologies that allow the traceability of the treatments to be determined and thus not incur the problems described above. This requires the use of methodologies that allow exhaustive monitoring of the traceability of the treatment, where the treating physician knows the origin of the drug and also has the ability of obtaining information on its pharmacokinetics and bioavailability. Among the strategies that can be used to follow the traceability of the treatment is the use of drugs labeled with heavy isotopes.

There are three main uses of isotopes in clinical pharmacology: (1) determining the pharmacokinetic profile and mechanism of action of a drug; (2) studying of the products of metabolization and sustained release systems to determine bioavailability and release profiles, respectively; and (3) evaluating patient-specific treatment relationships, for example evaluating pharmacokinetics to determine dosages, known as personalized medicine (Reunout et al.).

The use of stable isotopes for drug labeling plays an important role as a tool for a better understanding of drug metabolism. Stable isotopes are widely used in research and development as markers that allow the identification of metabolization sites and elucidate reaction mechanisms at an industrial level. In addition, they can be used as trackers that allow for monitoring and identification of the administered drug and its metabolites. The use of stable isotopes in these areas promotes the expansion of understanding drug metabolism and elucidation of reaction mechanisms. At the same time, a deep understanding of how a new molecule is metabolized and the knowledge of the distribution and toxicity of the drug related materials are crucial for the design of safe and effective drugs (Johnson, K.).

Stable isotopes represent different variants of the same chemical element that differ in their atomic weight and present differences in their physical and chemical properties. The isotopes generate changes in behavior, which is known as the isotope effect. WO 2020/245133 A1 and WO 2021/089872 A1 disclose deuterated DMT analogs to treat psychiatric or psycho-cognitive disorders. However, the large difference in atomic mass between hydrogen ¹H and its heavy isotope, deuterium ²H, results in the greatest isotope effect observed. In contrast, the use of stable heavy isotopes of carbon (carbon-13, ¹³C) and nitrogen (nitrogen-15, ¹⁵N) are not expected to generate relevant changes in the pharmacological behavior of the molecules that contain them because they do not present a significant isotope effect. Therefore, drugs that have these heavy isotopes (i.e. ¹³C and/or ¹⁵N) would not produce significant changes in the action or efficiency of the drug (Zachleder et al.).

For example, ES2320085 describes a method for labeling and identifying manufactured objects, substances and organisms based on the addition to the product to be labeled in a known mixture of two or more isotopic profiles of the same enriched chemical element in different stable isotopes. U.S. Pat. Nos. 5,760,394 and 7,112,445 teach a form of labeling of substances or products based on the addition of a tracer containing at least two elements, each possessing a minimum of two stable isotopes, with an artificial isotopic abundance ratio. EP1677105 describes the isotopic labeling of products using one or more organic compounds enriched in deuterium, ¹³C, ¹⁸O or ¹⁵N. This marking procedure is useful since it uses organic compounds that are originally in the composition of the product to be labeled and their isotopic abundance is modified by the addition of the marked compound. However, this type of marking could only be applied to products that do not undergo chemical reactions during their use since tracers could disappear.

WO 2021/030571 A1 describes methods for preventing or treating psychological disorders by administering serotonin receptor agonists separately, sequentially or simultaneously in combination with a 2A receptor antagonist. WO 2019/081764 A1 discloses a combination product with psychedelic effects for the treatment and/or prevention of psychiatric and/or neurological disorders. However, neither disclosure considers personalized dosage regimens for the individual.

The present invention relates to isotopic marking of classic tryptamine psychedelics that can have several beneficial applications. For example, isotopically labeled drugs can encode the product of mark its origin to ensure its traceability, prevent its falsification or detect its illicit use in the pharmaceutical, plant, food and drug abuse sectors, and to devise personalized treatments for individuals, among others. Because the major metabolite of DMT is IAA, in order to follow the pharmacokinetics it is beneficial to include marking not only in the alkyl chain, but also in the indole ring of DMT. The isotopically labeled tryptamine analogs disclosed herein have the same or equivalent pharmacological effect of the unlabeled analogs due to the small isotopic effect of ¹³C and/or ¹⁵N.

In an embodiment of the invention, the isotopically labeled tryptamine analogs are of formula (I) shown below:

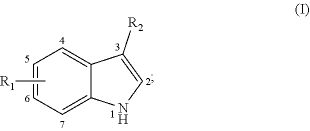

In formula (I), R₁ is selected from H, OH, PO₄H₂, OCH₃, and SCH₃; R₂ is selected from H and

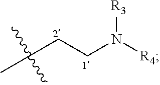

R₃ and R₄ are independently selected from H and CH₃; and the compound of formula (I) has one or more of a ¹⁵N atom and/or a ¹³C atom. In exemplary embodiments, R₃ and R₄ are methyl and R₁ is H. In other exemplary embodiments, R₃ and R₄ are methyl and R₁ is 5-methoxy.

In an embodiment of the invention, the isotopically labeled tryptamine analogs are of formula (Ia) shown below:

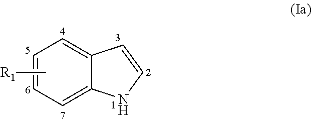

In formula (Ia), R₁ is selected from H, OH, PO₄H₂, OCH₃, and SCH₃; and the compound of formula (Ia) has one or more of a ¹⁵N atom at position 1, a ¹³C atom at position 2, and a ¹³C atom at position 3.

In an embodiment of the invention, the isotopically labeled tryptamine analogs are of formula (Ib) shown below:

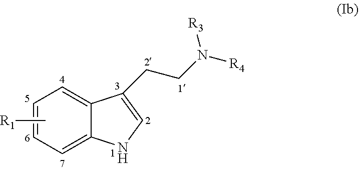

In formula (Ib) R₁ is selected from H, OH, PO₄H₂, OCH₃, and SCH₃; R₃ and R₄ are independently selected from H and CH₃; and the compound of formula (Ib) has one or more of a ¹⁵N atom at position 1, a ¹⁵N atom attached to position 1′, a ¹³C atom at position 1′, a ¹³C atom at position 2′, a ¹³C atom at position 2, and a ¹³C atom at position 3.

For isotopically labeled DMT, R₁ is H, and R₃ and R₄ are each CH₃ in formula (Ib). For isotopically labeled 5-MeO-DMT, R₁ is 5-MeO, and R₃ and R₄ are each CH₃ in formula (Ib).

Preferably the compound of formula (Ib) has one or more of a ¹⁵N atom at position 1 and a ¹⁵N atom attached to position 1′; or the compound of formula (Ib) has one or more of a ¹³C atom at position 2′; a ¹³C atom at position 2; and a ¹³C atom at position 3.

The isotopically labeled tryptamine analogs of formula (I), (Ia), and (Ib) can be either the free compounds or a pharmaceutically acceptable salt thereof, such as the fumarate salt.

Pharmaceutical compositions containing DMT and/or 5-MeO-DMT of natural abundance in combination with compounds of formula (Ib) are also disclosed. The pharmaceutical compositions comprise 1% or more, 10% or more, 30% or more, or 50% or more of compounds of formula (Ib).

Pharmaceutical compositions include, but are not limited to, tablets, capsules, pills, solutions, suspensions, nasal sprays, and powders. The pharmaceutical compositions can contain one or more pharmaceutically acceptable carriers or excipients.

### Preparation of Compounds of the Invention

The isotopic labeling of the compounds is carried out using, among others, the following organic synthesis methodologies. Methods are described to obtain the compounds proposed in this invention by specifying the positions marked with ¹³C and/or ¹⁵N. The atom numbering in the examples below is consistent with the atom numbering of formula (I), (Ia), and (Ib).

Depending on the compound to be synthesized, the indole derivative to be used will have a suitable substitution pattern for the desired product, and the amine used will be the appropriate one to obtain the product. Some of the possible combinations of labeled compounds are cited as examples, preferably including one or more labeled positions on the indole ring and/or on the alkyl amine chain.

To carry out the organic synthesis of Scheme 1 below, indole (2), or a suitable derivative thereof, is treated with oxalyl chloride, which affords compound 3, which then reacts with dimethylamine to afford the keto-amide compound (4), which undergoes reduction to provide DMT (Cozzi et al.). Exemplary sources of ¹³C labeled oxalyl chloride include, but are not limited to, oxalyl-¹³C₂ chloride available from Sigma Aldrich (98-99% purity). Exemplary sources of ¹³C labeled indole include, but are not limited to, indole 4-¹³C available from Cambridge Isotope Laboratories (95-99% purity).

Alternatively, indole (2) is reacted with N,N-dimethylethanolamine (5) in the presence of a ruthenium catalyst to afford DMT (Biswas et al.).

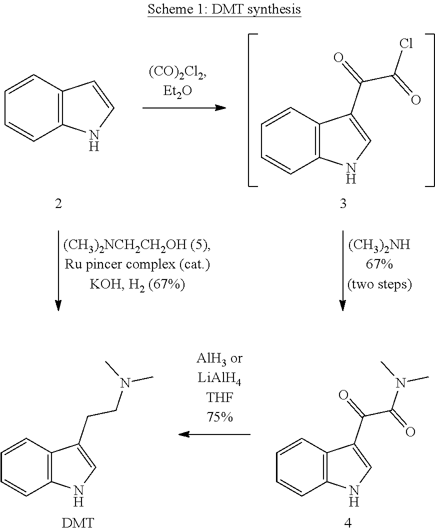

Based on the compound to be synthesized via scheme 1, the indole derivative used will have a substitution pattern suitable for the desired product, and the amine used will be that appropriate to obtain the desired product. Some of the possible combinations of labeled compound substrates are exemplified below, preferably including one or more labeled positions on the indole ring.
¹⁵N-labeled indole and ¹³C-labeled oxalyl chloride in one or more of the chain positions.
¹³C-labeled indole at C2 and ¹³C-labeled oxalyl chloride in one or more of the chain positions.
¹³C-labeled indole at C3 and ¹³C-labeled oxalyl chloride in one or more of the chain positions.
Indole labeled with ¹³C at C2 and dimethylamine labeled with ¹⁵N.
Indole labeled with ¹⁵N and oxalyl chloride labeled with ¹³C on both carbons.
Indole and ¹³C-labeled oxalyl chloride in one or more of the chain positions.

Another synthetic approach is the reductive amination of tryptamines of formula II to obtain the compound of formula (Ic), wherein R₁ is the same as that defined above, as shown in Scheme 2 below:

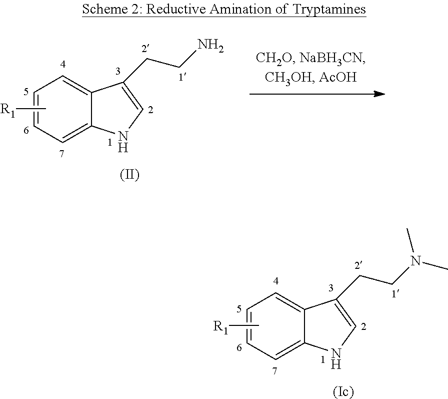

Based on the compound to be synthesized via scheme 2, the starting tryptamines of formula II contain stable ¹³C and/or ¹⁵N isotopes in specific positions and in an adequate abundance in order to generate identifiable patterns that allow determining the genuineness of the compounds and their origin. ¹³C and/or ¹⁵N labeled tryptamine can be obtained via decarboxylation of a tryptophan (Tanako et al.) of formula (III) (shown below):

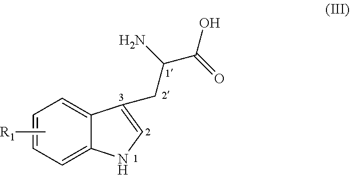

or from an indole (Noland et al.), suitably labeled with ¹³C and/or ¹⁵N. Exemplary sources of ¹³C and/or ¹⁵N labeled tryptamine include, but are not limited to, L-tryptophan (¹³C11, 99%; ¹⁵N2, 99%) available from Cambridge Isotope Laboratories.

Some non-limiting examples detailing the positioning of stable isotopes that would fulfill the aims sought in tryptamines for pharmacological or clinical use include the following:
Tryptamine of formula (II) that has a ¹⁵N atom in 1 position and a ¹³C atom in position 1′ and/or 2′.
Tryptamine of formula (II) that has a ¹⁵N atom in the 1 position and a ¹³C atom in the 2 and/or 3 position.
Tryptamine of formula (II) that has a ¹³C atom in the 2 and 3 position.
Tryptamine of formula (II) that has a ¹³C atom in 2 or 3 position and a ¹³C atom in position 1′ and/or 2′.
Tryptamine of formula (II) that has ¹³C atoms in position 2 and 3 and a ¹⁵N atom attached to carbon in position 1′.

The reductive amination approach is accomplished by the following synthetic procedure:
1. A compound of formula (II) (0.5 g) and acetic acid (0.9 ml) are dissolved in a solvent, such as methanol (49 ml), to obtain a first solution and cooling the first solution in an ice bath;
2. Adding sodium cyanoborohydride (0.39 g) and formaldehyde (0.66 ml) to the first solution to form a reaction mixture and stirring the reaction mixture for a predetermined amount of time, such as 5 hours;
3. Isolating the compound of formula (Ic).

To obtain a pharmaceutically acceptable salt of formula (Ic), the procedure can further include:
4. Dissolving the compound of formula (Ic) in a solvent, such as acetone, and adding a fumaric acid solution;
5. Isolating the fumarate salt of formula (Ic).

In embodiments, the first solution in step 1 is cooled in an ice bath and the solvent is methanol; in embodiments the predetermined amount of time in step 2 is 5 hours; in embodiments the solvent in step 4 is acetone and the fumaric acid solution is added at boiling temperature 6.

In embodiments, the isolating in step 3 encompasses the following;
a. Concentrating the reaction mixture under reduced pressure to obtain a reaction residue;
b. Diluting the reaction residue in an organic solvent to obtain a reaction residue solution;
c. Treating the reaction residue solution with NaOH (1M, 100 mL) to obtain an intermediate mixture;
d. Extracting the organic phase from the intermediate mixture with an organic solvent;
e. Repeating step d and combining all organic phases to obtain a combined organic phase;
f. Drying the combined organic phase with sodium sulfate;
g. Removing the organic solvent from the combined organic phase under reduced pressure to obtain the compound of formula (Ic).

In embodiments, the organic solvent in steps b, d, and g is methylene chloride

Yet another DMT synthetic strategy, shown in Scheme 3, is the reaction of phenyl hydrazine (6) and N,N-dimethylpropene-2-ene-1-amine (7) in the presence of a rhenium catalyst under a pressurized atmosphere of CO and H₂:

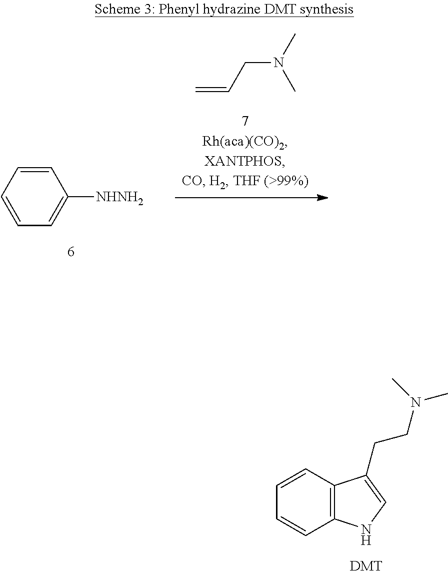

### Characterization of Isotopically Labeled Compounds

The stable isotope-labeled compounds can be characterized and detected through their molecular weight by different methods of mass spectrometry (MS), through different frequencies of their molecular vibrations by means of vibrational spectroscopy (e.g. Raman spectroscopy), or analyzed by different methods of nuclear magnetic resonance (i.e. ¹H or ¹³C NMR using DMSO-d₆ as the solvent) based on their nuclear spin. Infrared spectrum can be acquired in KBr pallets and/or using an ATR probe. Additionally, stable isotope-labeled compounds can be characterized and quantified using normal or high-resolution mass spectrometry (MS) and/or multi-stage mass spectrometry (MS/MS or Msⁿ), if necessary, in combination with separation techniques such as gas chromatography (GC) or high-pressure liquid chromatography (HPLC), sometimes including previous process of sample preparation and derivation (Schellekens et al.; Zachleder et al.; Wilkinson et al.).

### Compositions

Included in the present invention are various compositions that allow the use of isotopically labeled compounds to follow the drug and its metabolites after administration. These compositions include a compound marked with stable isotopes in an amount that is 1%, 10%, 30%, or 50%, or greater than or equal to 1%, 10%, 30%, or 50%, in one or more positions of the indole ring and/or in the side alkyl chain. The compositions thus contain a mixture, in defined proportions, of the unmarked and labeled compounds according to the abundance of isotope-labeled molecules ¹³C and/or ¹⁵N.

### REFERENCES

1. Baker et al., Front. Neurosci, 2018, 12: 536: “N, N-Dimethyltryptamine (DMT), an endogenous hallucinogen: past, present, and future research to determine its role and function.”

2. Bogenschutz et al., Journal of Psychopharmacology, 2015, 29 (3):289-99: “Psilocybin-Assisted Treatment for Alcohol Dependence: A Proof-of-Concept Study.”

3. Biswas et al. Adv. Synth. Catal. 2020, 362, 2902.

4. Callaway, J. C., Journal of Psychoactive Drugs, 2005, 37 (2): 157-61: “Fast and Slow Metabolizers of Hoasca.”

5. Chi, T. A., Journal of the Neurological Sciences, 2020, 411:116715: “Review of emerging therapeutic potential of psycodelic drugs in the treatment of psychiatric illnesses.”

6. Cozzi et al., Drug Test Anal, 2020, 12: 1483: “Synthesis and characterization of high-purity N, N-dimethyltryptamine (DMT) hemi fumarate for human clinical trials.”

7. David E. N., Pharmacological Reviews, 2016, 68 (2) 264-355: “Psychedelics.”

8. Davis et al. (1), The American Journal of Drug and Alcohol Abuse, 2019, 45(2):161-69: “5-Methoxy-N, N-Dimethyltryptamine (5-MeO-DMT) Used in a Naturalistic Group Setting Is Associated with Unintended Improvements in Depression and Anxiety.”

9. Garcia-Romeu et al., Current Drug Abuse Reviews, 2015, 7 (3):157-64: “Psilocybin-Occasioned Mystical Experiences in the Treatment of Tobacco Addiction.”

10. Luna, L. E., The Ethnopharmacology of Ayahuasca, 2011, 2: 01-21: “Indigenous and Mestizo Use of Ayahuasca: An Overview.”

11. Johnson, K., Identification and Quantification of Drugs, Metabolites, Drug Metabolizing Enzymes, and Transporters, 2020, Cap 15: 439: “The use of stable isotopes in drug metabolism studies.”

12. Johnson et al., The American Journal of Drug and Alcohol Abuse, 2017, 43:1, 55-60: “Long-term follow-up of psilocybin-facilitated smoking cessation.”

13. Kaasik et al., Journal of Psychoactive Drugs, 2020, 53 (1): 65-75: “Chemical Composition of Traditional and Analog Ayahuasca.”

14. Lima, O. G., Archives of the Institute of Agronomic Research, 1946, 4: 45-86: “Observations Over O ‘Vinho Da Jurema’ (Wine of Jurema) Used by the Pancarú Indian from Tacaratu (Pernambuco).”

15. Mckenna et al., Journal of Ethnopharmacology, 1984, 10 (2): 195-223: “Monoamine-Oxidase Inhibitors in South-American Hallucinogenic Plants-Tryptamine and Beta Carboline Constituents of Ayahuasca.”

16. Nichols et al., Handbook of Behavioral Neurobiology of Serotonin, 2020, Ed 2, Cap 49:959: “Classic psychedelics as therapeutics for psychiatric disorders.”

17. Noland et al. JACS, 1954, 76(12), 3227-3228.

18. Osório, et al., Brazilian Magazine of Psychiatry, 2015, 37(1):13-20: “Antidepressant Effects of a Single Dose of Ayahuasca in Patients with Recurrent Depression: A Preliminary Report.”

19. Palhano-Fontes et al., Psychological Medicine, 2019, 49 (4):655-63 “Rapid Antidepressant Effects of the Psychedelic Ayahuasca in Treatment-Resistant Depression: A Randomized Placebo-Controlled Trial.”

20. Panteghini, M., Clinical Biochemistry, 2009, 42:236-240: “Traceability as a unique tool to improve standardization in laboratory medicine.”

21. Reunout et al., Br. J. Clin Pharmacol, 2011, 72: 879: “Applications of stable isotopes in clinical pharmacology.”

22. Riba et al., The Journal of Pharmacology and Experimental Therapeutics, 2003, 306 (1): 73-83: “Human Pharmacology of Ayahuasca: Subjective and Cardiovascular Effects, Monoamine Metabolite Excretion, and Pharmacokinetics.”

23. Sanches et al., Journal of Clinical Psychopharmacology, 2016, 36 (1):77-81: “Antidepressant Effects of a Single Dose of Ayahuasca in Patients With Recurrent Depression: A SPECT Study.”

24. Schellekens et al., British Journal of Clinical Pharmacology, 2011, 72(6): 879-897: “Applications of stable isotopes in clinical pharmacology.”

25. Tanako et al., Heterocycles, 1977, 6(8), 1167-71.

26. Wilkinson et al., Clinical Nutrition Open Science, 2021, 36: 111-125: “Principles of stable isotope research—with special reference to protein metabolism.”

27. Zachleder et al., Biotechnology Advances, 2018, 36:784: “Stable isotope compounds—production, detection, and application.”

What is claimed:

1. A compound of formula (I) 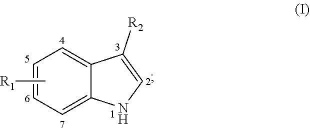
wherein R₁ is selected from the group consisting of H, OH, PO₄H₂, OCH₃, and SCH₃;
wherein R₂ is selected from the group consisting of H and 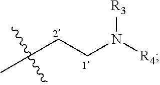
and
wherein R₃ and R₄ are independently selected from the group consisting of H and CH₃;
or a salt thereof;
wherein the compound of formula (I) comprises one or more of a ¹⁵N atom and/or a ¹³C atom.

2. The compound of claim 1, wherein the compound is a compound of formula (Ia) 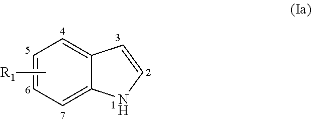
or a salt thereof;
wherein the compound of formula (Ia) comprises one or more of a ¹⁵N atom at position 1; a ¹³C atom at position 2; and a ¹³C atom at position 3.

3. The compound of claim 1 wherein the compound is a compound of formula (Ib) 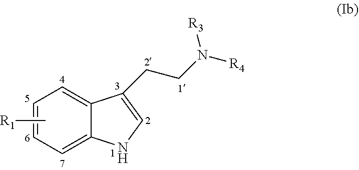
or a salt thereof;
wherein the compound of formula (Ib) comprises one or more of a ¹⁵N atom at position 1; a ¹⁵N atom attached to position 1′; a ¹³C atom at position 1′; a ¹³C atom at position 2′; a ¹³C atom at position 2; and a ¹³C atom at position 3.

4. The compound of claim 3, wherein R₁ is H, and R_{3,} and R₄ are each CH₃.

5. The compound of claim 3, wherein R₁ is 5-MeO, and R₃ and R₄ are each CH₃.

6. The compound of claim 4 or 5, comprising one or more of a ¹⁵N atom at position 1 and a ¹⁵N atom attached to position 1′.

7. The compound of claim 4 or 5, wherein comprising one or more of a ¹³C atom at position 2′; a ¹³C atom at position 2; and a ¹³C atom at position 3.

8. A composition comprising:
i) N,N-dimethyltryptamine (DMT) or 5-methoxy-N,N-dimethyltryptamine (5-MeO-DMT); and
ii) a compound of formula (Ib) 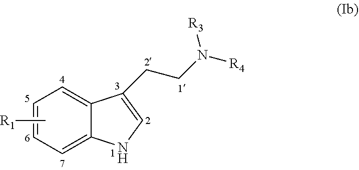
or a salt thereof;
wherein R₁ is selected from the group consisting of H, OH, PO₄H₂, OCH₃, and SCH₃;
wherein R₃ and R₄ are independently selected from the group consisting of H and CH₃; and
wherein the compound of formula (Ib) comprises one or more of a ¹⁵N atom at position 1; a ¹⁵N atom attached to position 1′; a ¹³C atom at position 1′; a ¹³C atom at position 2′; a ¹³C atom at position 2; and a ¹³C atom at position 3.

9. The composition of claim 8, comprising 1% or more, 10% or more, 30% or more, or 50% or more of the compound formula (Ib).

10. The composition of claim 8 or claim 9, wherein the compound of formula (Ib) is selected from the group consisting of a compound having:
a) R₁ is H, and R₃, and R₄ are each CH₃ and one or more of a ¹⁵N atom at position 1 and a ¹⁵N atom attached to position 1′;
b) R₁ is H, and R₃, and R₄ are each CH₃ and one or more of a ¹³C atom at position 1′; a ¹³C atom at position 2′; a ¹³C atom at position 2; and a ¹³C atom at position 3;
c) R₁ is 5-MeO, and R₃ and R₄ are each CH₃ and one or more of a ¹⁵N atom at position 1 and a ¹⁵N atom attached to position 1′; and
d) R₁ is 5-MeO, and R₃ and R₄ are each CH₃ and one or more of a ¹³C atom at position 1′; a ¹³C atom at position 2′; a ¹³C atom at position 2; and a ¹³C atom at position 3;

11. The composition of claim 8, comprising DMT and wherein the compound of formula (Ib) is selected from the group consisting of a compound having:
a) R₁ is H, and R₃, and R₄ are each CH₃ and one or more of a ¹⁵N atom at position 1 and a ¹⁵N atom attached to position 1′; and
b) R₁ is H, and R₃, and R₄ are each CH₃ and one or more of a ¹³C atom at position 1′; a ¹³C atom at position 2′; a ¹³C atom at position 2; and a ¹³C atom at position 3;.

12. The composition of claim 8 or claim 9, comprising 5-MeO-DMT and wherein the compound of formula (Ib) is selected from the group consisting of a compound having
c) R₁ is 5-MeO, and R₃ and R₄ are each CH₃ and one or more of a ¹⁵N atom at position 1 and a ¹⁵N atom attached to position 1′; and
d) R₁ is 5-MeO, and R₃ and R₄ are each CH₃ and one or more of a ¹³C atom at position 1′; a ¹³C atom at position 2′; a ¹³C atom at position 2; and a ¹³C atom at position 3.

13. A method of preparing a compound of formula (Ic) 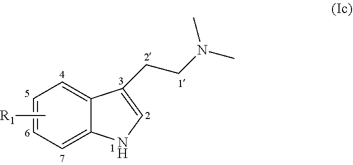
or a salt thereof,
the method comprising performing a reductive amination reaction on a compound of formula (II) 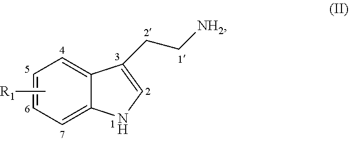
wherein R₁ is selected from the group consisting of H, OH, PO₄H₂, OCH₃, and SCH₃; and
wherein the compound of formula (Ic) comprises one or more of a ¹⁵N atom and/or a ¹³C atom.

14. The method of claim 13, wherein the reductive amination reaction comprises:
i) dissolving the compound of formula (II) and acetic acid in a solvent to form a first solution;
ii) adding to said first solution sodium cyanoborohydride and formaldehyde to form a reaction mixture; and
iii) isolating the compound of formula (Ic).

15. The method of claim 14, further comprising:
iv) dissolving the compound of formula (Ic) in a solvent and adding fumaric acid; and
v) isolating the fumarate salt of formula (Ic).

16. The method of claim 13, wherein the compound of formula (II) is selected from a compound having
a ¹⁵N label at position 1 and a ¹³C-label at position 1′ and/or position 2′;
a ¹³C-label at position C2 and a ¹³C-label at position 1′ and/or position 2′;
a ¹³C-labeled at position C3 and a ¹³C-label at position 1′ and/or position 2′;
a ¹³C-label at position C2 and a ¹⁵N label at the dimethyl substituted N; and
a ¹³C-label at position 1′ and/or position 2′.

17. The method of claim 13, wherein the compound of formula 2 is prepared from a tryptophan of formula (III)

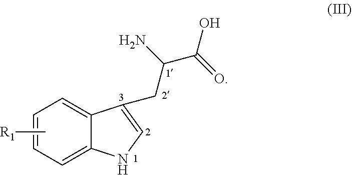

18. The method of claim 17, wherein the tryptophan comprises one or more of a ¹⁵N atom at position 1; a ¹⁵N atom attached to position 1′; a ¹³C atom at position 1′; a ¹³C atom at position 2′; a ¹³C atom at position 2; and a ¹³C atom at position 3.
